(19) United States
(12) Patent Application Publication
(10) Pub. No.: US 2020/0013017 A1
(43) Pub. Date: Jan. 9, 2020

(54) EMPLOYEE HEALTH ANALYTICS PROCESSOR

(71) Applicant: ADP, LLC, Roseland, NJ (US)

(72) Inventors: ROBERTO DIAS, PORTO ALEGRE (BR); JULIANA BEBER, PORTO ALEGRE (BR); GUILHERME GOMES, PORTO ALEGRE (BR); FABIANO PEREIRA, PORTO ALEGRE (BR); JULIO HARTMANN, PORTO ALEGRE (BR)

(21) Appl. No.: 16/026,099

(22) Filed: Jul. 3, 2018

(51) Int. Cl. G06Q 10/10; G06N 7/00; G16H 50/50

(52) U.S. Cl. CPC G06Q 10/105; G16H 50/50; G06N 7/005

(57) ABSTRACT

Aspects of the present invention provide devices that analyze employee health by determining a probability from reported measures of variances to normal work hours for at least one measure of expected employee health that includes expected sick leave or expected termination, and displaying the determined probability of expected employee health on a display device.

### BACKGROUND

The field of Human Capital Management (HCM) includes an entity monitoring employee work hours. The entity, such as a company, a non-profit organization, a business, a partnership, a corporation, and the like, employs the employees. The monitoring includes variances to work hours due to other activities, such as vacation, overtime, sick leave, and doctor appointments.

A conventional approach to monitoring of employee work hours and variances utilize computer systems, such as time reporting systems, human resource systems, payroll systems, etc., which record variances to normal work hours. The entity can use the reported information to ensure compliance with statutes, contracts, entity policies, and combinations thereof.

For example, monitoring of the reported overtime can include ensure proper compensation, adequate budget support, compliance with union contracts, and statutes governing overtime. Monitoring of vacation can include that only time agreed upon is taken for vacation by entity policy, employment contract, union rules, and combinations thereof. Monitoring of sick leave includes monitoring that the amount of time taken for sick leave does not exceed entity policy, such as benefits, and is proper under law. Each variance is separately monitored to ensure compliance.

### BRIEF SUMMARY

In one aspect of the present invention, a computer-implemented method for analyzing employee health includes executing on a computer processor determining a probability from reported measures of variances to normal work hours for at least one measure of expected employee health selected from a group consisting of expected sick leave and expected employment termination, and displaying the determined probability of expected employee health on a display device.

In another aspect, a system has a hardware processor, computer readable memory in circuit communication with the processor, and a computer-readable storage medium in circuit communication with the processor and having program instructions stored thereon. The processor executes the program instructions stored on the computer-readable storage medium via the computer readable memory and thereby analyzing employee health, which determines a probability from reported measures of variances to normal work hours for at least one measure of expected employee health selected from a group consisting of expected sick leave and expected employment termination, and displays the determined probability of expected employee health on a display device.

In another aspect, a computer program product for analyzing employee health has a computer-readable storage medium with computer readable program code embodied therewith. The computer readable hardware medium is not a transitory signal per se. The computer readable program code includes instructions for execution by a processor that cause the processor to determine a probability from reported measures of variances to normal work hours for at least one measure of expected employee health selected from a group consisting of expected sick leave and expected employment termination, and display the determined probability of expected employee health on a display device.

### BRIEF DESCRIPTION OF THE DRAWINGS

These and other features of embodiments of the present invention will be more readily understood from the following detailed description of the various aspects of the invention taken in conjunction with the accompanying drawings in which:

FIG. 1 depicts a schematic illustration of system aspects according to an embodiment of the present invention.

FIG. 2 is a flow chart illustration of an embodiment of the present invention.

FIG. 3 depicts an example user interface according to an embodiment of the present invention.

FIG. 4 depicts an example user interface according to an embodiment of the present invention.

### DETAILED DESCRIPTION

The present invention may be a system, a method, a computer program product, and combinations thereof. The computer program product may include a computer readable storage medium (or media) having computer readable program instructions thereon for causing a processor to carry out aspects of the present invention.

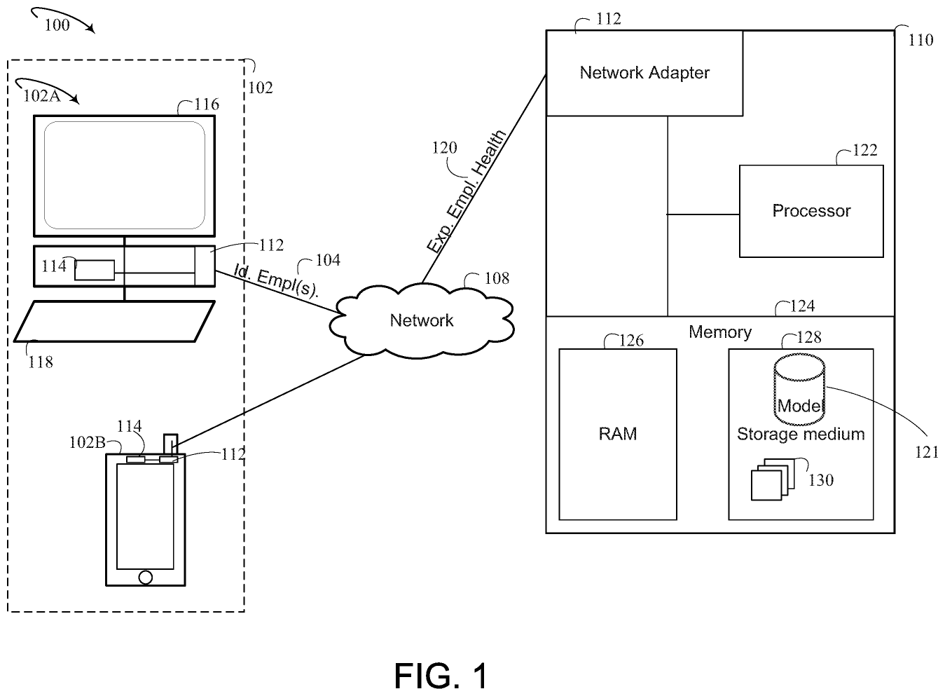

With reference to FIG. 1, a schematic of an embodiment of a system 100 for analyzing employee health is depicted. The system 100 includes a local computing device 102, such as, for example, a desktop computer 102A, laptop computer, personal digital assistant, tablet, smartphone 102B, cellular telephone, body worn device, and the like.

The local computing device 102 identifies one or more employees 104 of an entity. The identified employees 104 can be grouped by team, managerial reporting relationship, department, entity, and combinations thereof. In some embodiments, the identification can be based on a logon of a user. For example, a manager A logs into the system 100 and a corresponding user profile identifies the employees reporting to manager A.

The local computing device 102 transfers the identified employee 104 over a network 108 to a computer server 110. The identified employee 104 can be represented as an alphanumeric character string, such as an employee identifier, an entity identifier, a department identifier, a team identifier, a manager identifier, and the like. The local computing device 102 includes a network interface adapter 112, a processor 114, a display device 116 and one or more input devices 118, such as a keyboard, touch screen, mouse, microphone, and the like. The local computing device 102 can include displays on the display device 116 and inputs from the input devices 118 to identify the employee 104 through a user interface and transfer the identified employee 104 to the computer server 110.

The computer server 110, in response to the identified employee 104, selects reported measures of variances to normal work hours corresponding to the identified 104. The measures can include employee work hours, employee overtime, employee prior sick leave, employee medical appointment, employee vacation, and combinations thereof. The measures can include further attributes of a job title indicator, a working hours indicator, a company size indicator, a location indicator, an industry indicator, an employee age indicator, and combinations thereof.

The computer server 110 determines a probability for a measure of expected employee health 120. The expected employee health 120 can include expected sick leave, expected termination, and combinations thereof. The expected sick leave includes days in which the employee is not able to work due to illness. The expected termination includes employee separation from the entity. For example, the employee leaves employment by the entity.

The expected employee health 120 includes a probability for one or more employees. The probability can be determined according to a model 121, which can include a statistical model, a deep learning model and combinations thereof. In some embodiments, the expected employee health 120 includes a probability for a group of employees.

The computer server 110 returns the expected employee health 120 to the local computing device 102, which displays the expected employee health 120 on the display device 116. The displayed expected employee health 120 can include a dashboard display or a portion thereof.

The expected employee health 120 strategically changes analysis of employee health from the conventional practice of ensuring compliance of individual retrospective measures to viewing employee health as interrelated measures that can lead to non-productive variances to normal work hours. For example, in some instances excessive overtime, lack of vacation, medical appointments can be predictors of future sick leave, employment termination, and combinations thereof. The expected employee health 120 can provide a prospective outlook of employment, rather than a retrospective view of compliance. The expected employee health 120 can help avoid non-productive sick leave, which can interrupt team efforts. The expected employee health 120 can be indicative of a probable loss of the employee due to termination, which can be a permanent loss of a skilled resource. The expected employee health 120 can help the entity utilize the employee as a healthy productive asset by identifying potential non-productive situations that can be managed.

The lines of the schematic illustrate communication paths between devices and between components with each device. Communication paths between the local computing device 102 and the computer server 110 over the network 108 include a network interface device 112 in each device, such as a network adapter, network interface card, wireless network adapter, and the like.

The computer server 110 includes a processor 122 configured with instructions stored in a memory 124. The processor 122 of the computer server 110 and the processor 114 of the local computing device include, for example, a digital processor, an electrical processor, an optical processor, a microprocessor, a single core processor, a multi-core processor, distributed processors, parallel processors, clustered processors, combinations thereof and the like. The memory 124 includes a computer readable memory 126 and a computer readable storage medium 128.

The computer readable storage medium 128 can be a tangible device that retains and stores instructions for use by an instruction execution device, such as the processor 122. The computer readable storage medium 128 may be, for example, but is not limited to, an electronic storage device, a magnetic storage device, an optical storage device, an electromagnetic storage device, a semiconductor storage device, or any suitable combination of the foregoing. A computer readable storage medium 128, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be transmitted to respective computing/processing devices from the computer readable storage medium 128 or to an external computer or external storage device via the network 108. The network 108 can include private networks, public networks, wired networks, wireless networks, data networks, cellular networks, local area networks, wide area networks, the Internet, and combinations thereof. The network interface device 112 in each device receives computer readable program instructions from the network 108 and forwards the computer readable program instructions for storage in the computer readable storage medium 128 within the respective computing/processing device.

Computer readable program instructions for carrying out operations of the present invention may include assembler instructions, instruction-set-architecture (ISA) instructions, machine instructions, machine dependent instructions, microcode, firmware instructions, state-setting data, configuration data for integrated circuitry, compiled or interpreted instructions, source code or object code written in any combination of one or more programming languages or programming environments, such as Java® (Java is a registered trademark of Oracle America, Inc.), Javascript, C, C#, C++, Python, Cython, F#, PHP, HTML, Ruby, and the like.

The computer readable program instructions may execute entirely on the computer server 110, partly on the computer server 110, as a stand-alone software package, partly on the computer server 110 and partly on the local computing device 102 or entirely on the local computing device 102. For example, the local computing device 102 can include a web browser that executes HTML instructions transmitted from the computer server 110, and the computer server executes Java® instructions that construct the HTML instructions. In another example, the local computing device 102 includes a smartphone application, which includes computer readable program instructions to perform identifying and transfer of the identified employee 104, and the computer server 110 includes different computer readable program instruction to receive the identified employee 104 and determine the expected employee health 120.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine (“a configured processor”), such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The memory 124 can include a variety of computer system readable media. Such media may be any available media that is accessible by computer server 110, and the media includes volatile media, non-volatile media, removable, non-removable media, and combinations thereof. Examples of the volatile media can include random access memory (RAM) and/or cache memory. Examples of non-volatile memory include magnetic disk storage, optical storage, solid state storage, and the like. As will be further depicted and described below, the memory 124 can include at least one program product having a set (e.g., at least one) of program modules 130 that are configured to carry out the functions of embodiments of the invention.

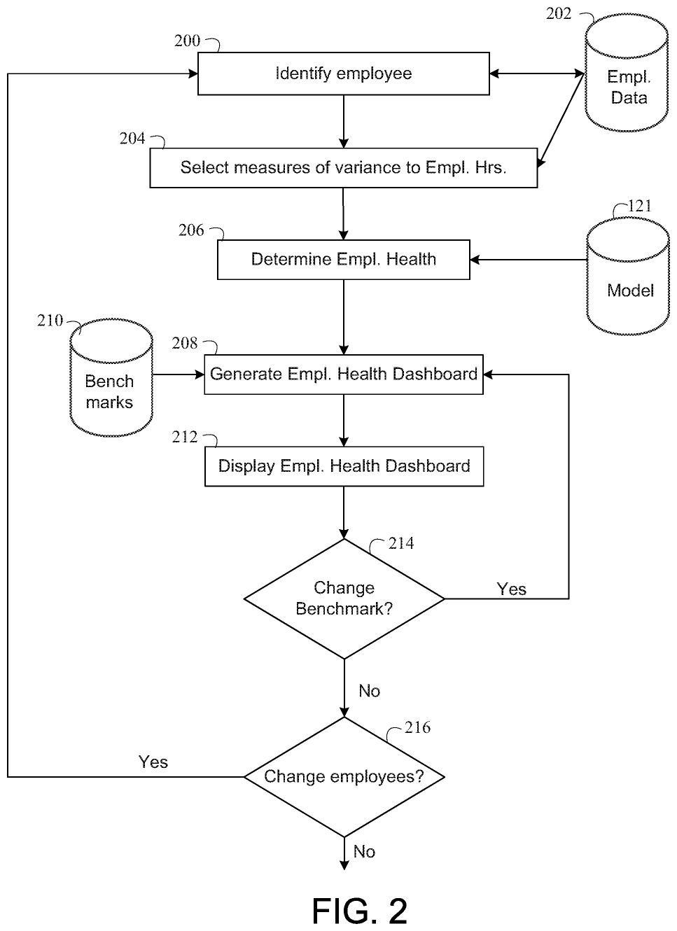

FIG. 2 illustrates one embodiment of a method according to the present invention for analyzing employee health. At 200, a processor that is configured according to an aspect of the present invention (the “configured processor”) identifies the employee 104 or group of employees. The identified employee 104 can be identified according to an input from the input device 118 (FIG. 1). The identified employee 104 can be identified according to a user profile or login identifier. The identified employee 104 can input data from an employee database 202, such as a personnel database, human resource database, payroll database, and the like. The identified employee can include a group of employees, such as a team, a managerial reporting relationship, a department, an entity, and combinations thereof.

At 204, the configured processor selects measures of variances to normal work hours for the identified employee 104. Normal work hours can include a standard business work week, such as eight (8) hour shift, Monday through Friday. In some embodiments, normal work hours are determined according to the employee position, such as first shift, second shift, third shift, alternate work days, alternate work schedules, and the like.

The measures of variances to normal work hours include vacation, overtime, prior sick leave, medical appointment, and combinations thereof. The measures of variances to normal work hours can include other attributes, such as the job title indicator, the working hours indicator, the company size indicator, the location indicator, the industry indicator, the employee age indicator, and combinations thereof. The measures of variances to normal work hours are reported variances, such as recorded in an employee time reporting system, human resource system, payroll system, and the like. Measures can include day or hour measures. Measures can include a number of occurrences. Measures include reporting periods, such as daily, weekly, monthly, yearly, and combinations thereof. For example, measures of reported vacation can include 4.5 days in a specified week, or alternatively as 2 occurrences totaling 36 hours in the month of June.

For example, reported vacation includes numerical values of vacation taken, which can include days or hours spent not working during the normal work week. Reported overtime, for example, can include additional hours worked over normal daily and/or weekly hours, whether compensated or non-compensated. The reported sick leave can include days not working during the normal work week due to illness, whether compensated or non-compensated, whether formal leave or informal sick days. In some embodiments, sick leave is identified as a minimum number of contiguous days, such as, for example, 3 days, and medically approved. The reported medical appointment can include, for example, a number of occurrences, a number of days, a and/or a number of hours away from normal work hours due medical reasons, such as a doctor visit, a medical test, a medical examination, a medical procedure, a hospital stay, and the like. In some embodiments, sick leave, overtime, and/or medical appointment are defined by statute, by entity policy, and combinations thereof.

At 206, the configured processor determines a probability for the expected employee health 120 from the reported measures of variances to normal work hours. In some embodiments the reported measures of variances to normal work hours includes the other attributes of employment. The expected employee health includes expected sick leave, expected employment termination, and combinations thereof. The configured processor uses the model 121 to determine the probability for expected employee health 120.

The model 121 can include a statistical model, such as an analysis of variance model, a linear regression model, a descriptive statistic model and a multivariate analysis model, and combinations thereof. For example, a linear regression model can include an independent variable of expected sick leave, and dependent variables of prior sick leave, overtime, medical appointment, company size indicator, location indicator, industry indicator, employee age indicator. In another example, a descriptive statistic, such as a mean, median, mode, standard deviation, correlation, quartile, quintile, and the like, indicate the probability of the expected sick leave in relation to a measure of the reported measures of variances to normal work hours, such as a 0.75 probability of a sick leave in the next six months for at least one member of the analyzed group of employees when compared a benchmark of employees with similar attributes.

Job title indicator can include a classification of the job title. For example, the job title of developer IV can be classified as a job type of information technology (IT). The job type can include roles, such as engineering, finance, marketing, sales, legal, IT, etc. The job title can be classified as a job level, such as managerial/non-managerial, or as a reporting level within the entity represented as an integer, which represents the number of levels to report to the highest position in the entity. For example, 0 for chief executive officer (CEO), 1 for chief operating officer (COO) reporting to CEO, 2 for manager reporting to COO, 3 for engineer reporting to manager, etc. Combinations of job type and job level are contemplated.

The working hours indicator can include a numerical quantity of hours, or a classification, such as a shift identifier, exempt/non-exempt, salaried/hourly, etc.

The company size indicator can be represented as a number of employees, an amount of revenue, a classified range of the number of employees, or a classified range of the amount of revenues.

The location indicator can be represented as a state code, a Zone Improvement Plan (ZIP) code, or groupings thereof.

The industry indicator indicates a type of industry in which the employee is employed. In some embodiments, the industry indicator can be based on industry classifications, such as North American Industry Classification System (NAICS), Industry Classification Benchmark (ICB), Standard International Trade Classification (SITC), and the like. In some embodiments, the industry indicator indicates the industry of the type of work performed by the employee, such as engineering, marketing, sales, etc. In some embodiments, values of the industry indicator are mapped from a plurality of industries to a single value of the industry indicator.

The employee age indicator can be represented as an age of the employee or a classification of the age of the employee into a range of values.

The model 121 can include a trained deep learning model, such as a deep neural network. For example, a deep neural network can be trained using a target of expected sick leave and a feature vector of the prior sick leave, overtime, medical appointment, job title indicator, working hours indicator, company size indicator, location indicator, industry indicator, employee age indicator, and combinations thereof. The trained deep learning model can classify attributes of one or more employees to target values of the expected employee health 120.

At 208, the configured processor generates a dashboard that includes the expected employee health 120. The generated dashboard can include benchmarks 210, such as teams within the same entity, teams within the same department, employees within the same entity, employees within the same department, employees within the same industry indicator, and the like. The benchmarks can include combinations of the prior sick leave, the overtime, the medical appointment, the job title indicator, the working hours indicator, the company size indicator, the location indicator, the industry indicator, and the employee age indicator. The dashboard can represent the probability and the benchmarks numerically, textually, graphically, or combinations thereof.

At 212, the configured processor displays the generated dashboard on the display device 116 (FIG. 1), which includes the expected employee health 120.

At 214, the configured processor, in response to an input from the input device 118 can change benchmarks. For example, a default for the generated display can include a first benchmark, such as other teams within a department of an entity, and the input can change the default benchmark to a second benchmark, such as other employees within a same industry indicator and a same location indicator.

At 216, the configured processor, in response to an input from the input device 118, can change the identified employee 104. For example, the input can identify employee, another team, another department, or another reporting level within the entity.

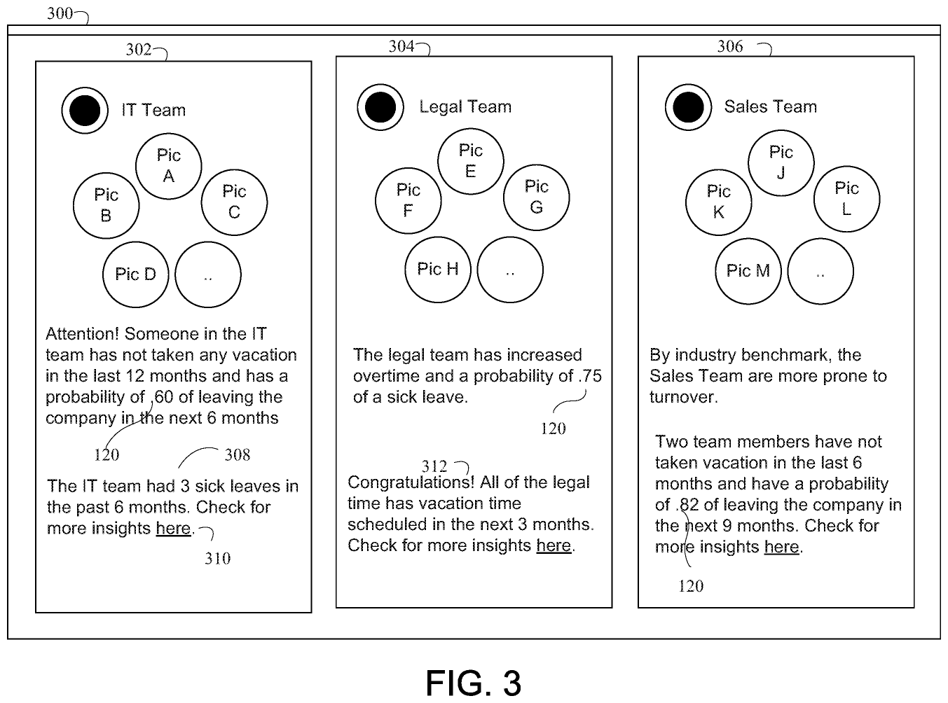

FIG. 3 depicts an example user interface according to an embodiment of the present invention, which displays a dashboard window 300 on the display device 116 with the expected employee health 120 for each of an IT team display 302, a legal team display 304, and a sales team display 306.

The expected employee health 120 for the IT team display 302 includes a probability of 0.60 of an employee termination indicating by the text “leaving the company in the next 6 next six months.” The expected employee health 120 indicates the variance to normal work hours as vacation, indicated by the text “someone in the IT team has not taken any vacation in the last 12 months.” The dashboard display can include further indicators of the expected employee health 120, such as text, for example, “Attention!”, color coding in red. The dashboard window 300 can include relevant reported measures of variance to normal work hours or other attributes 308, such as represented with the text “The IT team had 3 sick leaves in the past 6 months.” The dashboard window 300 can provide for further input 310, such as indicated by the text and hypertext link labeled “Check for more insights here.” Selecting the link using the input device 118, can provide the expected employee health 120 for each employee in the IT team, or compare the expected employee health 120 or the measures of variance to normal work hours with the benchmark 210, such as graphically with line graphs, bar charts, scatter plots, and the like.

The legal team display 304 includes the expected employee health 120, represented by the probability 0.75 and the text “of a sick leave,” indicating an expected sick leave. The measures of variance to normal work hours include overtime as indicated by the text “the legal team has increased overtime.” The expected employee health can include further indicators 312, which are positively reported and indicate a low probability. The text “Congratulations! All of the legal team has vacation time scheduled in the next 3 months,” represents a low probability for the measured variance to normal work hours, which includes overtime. The color coding for a positive indicator, for example, can be indicated in green.

The sales team display 306 includes the expected employee health 120, represented by the probability of 0.82 and text “leaving the company in the next 9 months.” The measures of variance to normal work hours are indicated by the text “two team members have not taken a vacation in the last 6 months.”

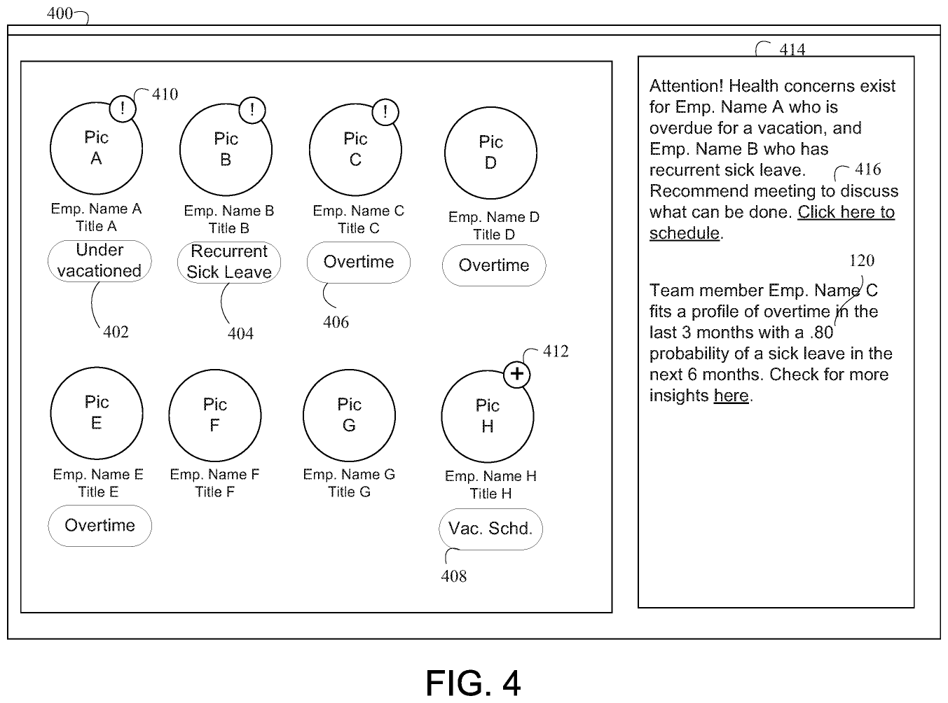

FIG. 4 depicts an example user interface according to an embodiment of the present invention, which displays another dashboard window 400 on the display device 116. The dashboard window 400 can be generated in response to an input, such as the input 310 described in reference to FIG. 3. For example, the dashboard window 400 can be representative of a view of the IT team, i.e. different reporting level, such as described in reference to act 216 of FIG. 2.

For a first employee, a first measure of variance to normal work hours 402 is indicated by the text “under vacationed,” which represents a classification of the vacation. For a second employee, a second measure of variance to normal work hours 404 is indicated by the text “recurrent sick leave,” which represents a classification of the prior sick leave. For a third employee, a third measure of variance to normal work hours 406 is indicated by the text “overtime,” which represents a classification of the overtime. For a fourth employee, a fourth measure of variance to normal work hours 408 is indicated by the text “Vac. Sched.,” which represents another classification of the vacation.

The variance measures of variance to normal work hours 402, 404, 406, 408 can include further indicators, which indicate low probability or high probability for expected employee health 120. For example, the first employee includes a first further indicator 410 of an encircled exclamation mark, which indicates a high probability for the expected employee health 120. That is, the measures of variance to normal work hours 402 for the first employee indicate an increased likelihood that the employee is likely to either have an expected sick leave or an expected termination. The fourth employee includes a second further indicator 412 of an encircled plus sign, which indicates a low probability for the expected employee health 120. That is, the measures of variance to normal work hours 408 for the employee indicate a likelihood that the employee is not likely to either have an expected sick leave or an expected termination.

The dashboard window 400 includes a second display 414, which includes an identification of the measures of variances to normal work hours contributing to the expected employee health 120 for the team. The identification includes a classification of the vacation represented by the text “overdue vacation” and a classification of the prior sick leave represented by the text “recurrent sick leave.” The second display 414 includes a corrective recommendation 416 to address the expected employee health 120, such as scheduling a meeting with the team or affected employee.

The second display 414 highlights employees with high probabilities for expected employee health 120. For example, the expected employee health 120 for “Empl. Name C” is indicated with a probability of 0.80 according to the model 121. The expected employee health 120 is indicated for expected sick leave with the text “a sick leave in the next 6 months.” The measures of variance to normal work hours that contribute to the high probability of 0.80 are indicated with a classification of the overtime indicated by the text “fits a profile of overtime in the last 3 months.”

The terminology used herein is for describing particular aspects only and is not intended to be limiting of the invention. As used herein, the singular forms “a”, “an” and “the” are intended to include the plural forms as well, unless the context clearly indicates otherwise. It will be further understood that the terms “include” and “including” when used in this specification specify the presence of stated features, integers, steps, operations, elements, and/or components, but do not preclude the presence or addition of one or more other features, integers, steps, operations, elements, components, and/or groups thereof. Certain examples and elements described in the present specification, including in the claims, and as illustrated in the figures, may be distinguished, or otherwise identified from others by unique adjectives (e.g. a “first” element distinguished from another “second” or “third” of a plurality of elements, a “primary” distinguished from a “secondary” one or “another” item, etc.) Such identifying adjectives are generally used to reduce confusion or uncertainty, and are not to be construed to limit the claims to any specific illustrated element or embodiment, or to imply any precedence, ordering or ranking of any claim elements, limitations, or process steps.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for analyzing employee health, comprising executing on a computer processor:
determining a probability from reported measures of variances to normal work hours for at least one measure of expected employee health selected from a group consisting of expected sick leave and expected employment termination; and
displaying the determined probability of expected employee health on a display device.

2. The method of claim 1, wherein the reported measures of variances to normal work hours comprise at least one measure selected from a group consisting of vacation, overtime, sick leave and medical appointment.

3. The method of claim 1, wherein the determining the probability for the at least one measure of expected employee health comprises modeling the reported measures of variances to normal work hours and the at least one measure of expected employee health using at least one statistical model selected from a group consisting of analysis of variance, linear regression, descriptive statistics and multivariate analysis.

4. The method of claim 1, wherein the determining the probability for the at least one measure of expected employee health comprises classifying reported measures of variances to normal work hours according to the at least one measure using a trained deep learning model.

5. The method of claim 1, wherein the reported measures of variances to normal work hours comprise at least one interval selected from a group consisting of a day, a week, a month, and a year.

6. The method of claim 1, wherein the determining the probability is based on a data set of entity-employee benchmark data.

7. The method of claim 6, wherein the data set of entity employee benchmark data comprises at least one attribute selected from a group consisting of entity size, entity location, entity industry, employee job title, and employee age.

8. The method of claim 6, wherein the data set of entity employee benchmark data comprises at least one attribute selected from a group consisting of employee work hours, employee overtime, employee sick leave, employee medical appointments, and employee vacation.

9. A system for analyzing employee health, comprising:
a processor;
a computer readable memory in circuit communication with the processor; and
a computer readable storage medium in circuit communication with the processor;
wherein the processor executes program instructions stored on the computer-readable storage medium via the computer readable memory and thereby:
determines a probability from reported measures of variances to normal work hours for at least one measure of expected employee health selected from a group consisting of expected sick leave and expected employment termination; and
displays the determined probability of expected employee health on a display device.

10. The system of claim 9, wherein the reported measures of variances to normal work hours comprise at least one measure selected from a group consisting of vacation, overtime, sick leave and medical appointment.

11. The system of claim 9, wherein the processor executes program instructions stored on the computer-readable storage medium via the computer readable memory and thereby:
models the reported measures of variances to normal work hours and the at least one measure of expected employee health using at least one statistical model selected from a group consisting of analysis of variance, linear regression, descriptive statistics and multivariate analysis.

12. The system of claim 9, wherein the processor executes program instructions stored on the computer-readable storage medium via the computer readable memory and thereby:
classifies reported measures of variances to normal work hours according to the at least one measure using a trained deep learning model.

13. The system of claim 9, wherein the reported measures of variances to normal work hours comprise at least one interval selected from a group consisting of a day, a week, a month, and a year.

14. The system of claim 9, wherein the determined probability is based on a data set of entity-employee benchmark data.

15. A computer program product for analyzing employee health, the computer program product comprising:
a computer readable storage medium having computer readable program code embodied therewith, wherein the computer readable storage medium is not a transitory signal per se, the computer readable program code comprising instructions for execution by a processor that causes the processor to:
determine a probability from reported measures of variances to normal work hours for at least one measure of expected employee health selected from a group consisting of expected sick leave and expected employment termination; and
display the determined probability of expected employee health on a display device.

16. The computer program product of claim 15, wherein the reported measures of variances to normal work hours comprise at least one measure selected from a group consisting of vacation, overtime, sick leave and medical appointment.

17. The computer program product of claim 15, wherein the instructions for execution cause the processor to:
model the reported measures of variances to normal work hours and the at least one measure of expected employee health using at least one statistical model selected from a group consisting of analysis of variance, linear regression, descriptive statistics and multivariate analysis.

18. The computer program product of claim 15, wherein the instructions for execution cause the processor to:
classify reported measures of variances to normal work hours according to the at least one measure using a trained deep learning model.

19. The computer program product of claim 15, wherein the reported measures of variances to normal work hours comprise at least one interval selected from a group consisting of a day, a week, a month, and a year.

20. The computer program product of claim 15, wherein the determined probability is based on a data set of entity-employee benchmark data.
